(19) United States
(12) Patent Application Publication
(10) Pub. No.: US 2026/0327541 A1
(43) Pub. Date: Oct. 22, 2026

(54) STRUCTURE TO WHICH MEMBRANE IS THERMALLY BONDED, AND METHOD FOR MANUFACTURING SAME

(71) Applicants: POSTECH RESEARCH AND BUSINESS DEVELOPMENT FOUNDATION, Pohang-si (KR); UNIST(ULSAN NATIONAL INSTITUTE OF SCIENCE AND TECHNOLOGY), Ulsan (KR)

(72) Inventors: Dong Sung KIM, Pohang-si (KR); Jae Seung YOUN, Pohang-si (KR); Jeong Won CHOI, Ulsan (KR); Tae Eun PARK, Ulsan (KR); Jun Yeol RHYOU, Seoul (KR)

(21) Appl. No.: 19/113,989

(22) PCT Filed: Aug. 22, 2023
(86) PCT No.: PCT/KR2023/012435

§ 371 Date: Mar. 21, 2025

(30) Foreign Application Priority Data
Sep. 23, 2023 (KR) 10-2022-0120730

(51) Int. Cl. C12M 1/12

(52) U.S. Cl. CPC C12M 25/02

(57) ABSTRACT

The present invention relates to a structure to which a membrane is thermally bonded, and a method for manufacturing same. More specifically, the present invention relates to a structure to which a membrane is thermally bonded, and a method for manufacturing same, the structure having a porous nanomembrane that is thermally bonded to a bonding body so that adhesiveness between the membrane and the bonding body is improved, and thus other impaired states (for example, bonding separation), which can occur in the analysis of experiments and research, are prevented or accommodation bodies (for example, culture cells), which are accommodated in the bonding body, are prevented, during experiments and research, from leaking to the outside of the bonding body.

### TECHNICAL FIELD

The present disclosure relates to a structure to which a membrane is thermally bonded and a method of manufacturing the structure. More specifically, the present disclosure relates to a structure to which a membrane is thermally bonded, and a method for manufacturing same, the structure having a porous nanomembrane that is thermally bonded to a bonding body so that adhesiveness between the membrane and the bonding body is improved, and thus other impaired states (for example, bonding separation), which can occur in the analysis of experiments and research, are prevented or accommodation bodies (for example, culture cells), which are accommodated in the bonding body, are prevented, during experiments and research, from leaking to the outside of the bonding body.

### BACKGROUND ART

A porous nanomembrane is a film constituted by intertwining a plurality of polymer fibers, and having a thickness of tens to hundreds of nanometers, with numerous permeation pores that selectively allow a particular substance to pass through.

The porous nanomembranes have characteristics such as a low cost, a simple process, transparency, and ease of adsorption and desorption to be used both inside and outside a body. In addition, since a size of permeation pores and a thickness of a membrane may be freely adjusted by controlling a type and concentration of solvents, the porous nanomembranes is known as having a high application value.

The porous nanomembranes are mainly used in cell culture, cell research and analysis. In this case, the porous nanomembranes function as cell culture surfaces in cell culture or cell membranes in cell research and analysis.

The porous nanomembranes need to be attached or bonded to a container containing cells to fully perform functions. However, nanomembrane bonding technologies in the related art are limited to methods of simply applying adhesives or attaching adhesive tapes (such as double-sided tape) to cell containers, which causes many problems in cell culture and cell research. For example the method of applying adhesives may have a negative effect on an experimental environment, such as slowing cell growth due to inherent substances of the adhesive, and there have been many problems such that the method of attaching an adhesive tape result in easy detachment over time or creation of gaps at a bonding surface to cause cells to leak out.

The present disclosure has been derived from the aforementioned problems, and is directed to solving the technical problems described above and other drawbacks, and also providing additional technical elements that cannot be easily devised by those skilled in the art.

### DISCLOSURE OF INVENTION

### Technical Problem

Therefore, the present disclosure has been made in view of the above problems, and it is an object of the present disclosure to provide a structure and a method of manufacturing the structure, the structure being configured to improve a bonding strength between a membrane and a bonding body and allow a bonding surface to be firmly maintained even after bonding, compared to an adhesive application method or an adhesive attachment method in the related art.

In addition, the present disclosure also provides a structure to which a membrane optimum for thermal bonding is thermally bonded, by applying heat to a bonding surface to thermally bond the membrane to a bonding body at a particular temperature at which a non-bonding surface of the membrane is not thermally deformed during bonding to the membrane as the heat is not transferred to the non-bonding surface.

The present disclosure may, however, be embodied in many different forms and should not be construed as being limited to the embodiments set forth herein. Additional aspects will be set forth in part in the description which follows and, in part, will be apparent from the description, or may be learned by practice of the presented exemplary embodiments.

### Technical Solution

To accomplish the above object, according to an embodiment of the present disclosure, there is provided a structure to which a membrane is bonded. At this time, the structure may include: the membrane made of a material of a nanofiber; and a bonding body which is a bonding target of the membrane. The membrane may include: a bonding surface constituted by being brought into contact with a bonding portion of the bonding body to which heat at a particular temperature is applied, and bonded to the bonding body by being thermally deformed due to the contact; and a permeation surface other than the bonding surface, the permeation surface having a porous structure.

In addition, in the structure to which the membrane is bonded, the particular temperature may be in a range from [a melting point Tm] to [the melting point Tm+10 °C.] of the membrane.

In addition, in the structure to which the membrane is bonded, the bonding surface may be a surface of which a permeability is reduced due to deformation of a structure of the nanofiber compared to a permeability function before the thermal deformation, and through which cells of fluids, or fluids are significantly limited from passing.

In addition, in the structure to which the membrane is bonded, the structure may have values of different ratios between a deformed permeation surface and a preserved permeation surface depending on a speed at which the bonding portion and the membrane approach each other.

Meanwhile, according to another embodiment of the present disclosure, there is also provided a method of manufacturing a structure to which a membrane is bonded, the method including: applying heat at a particular temperature to a bonding portion of a bonding body; and thermally bonding the bonding portion of the bonding body and the membrane by causing the bonding portion of the bonding body and the membrane to approach each other, wherein the membrane after the thermally bonding may include a bonding surface and a permeation surface, the bonding surface may be a surface constituted by being brought into contact with a bonding portion of the bonding body to which heat at a particular temperature is applied, and bonded to the bonding body by being thermally deformed due to the contact, and the permeation surface may be a surface other than the bonding surface, and has a porous structure.

### Advantageous Effects

According to the present disclosure as described above, there are such effects that a bonding strength between a membrane and a bonding body is enhanced and a bonding surface is maintained firmly even after bonding, and further, a damage to the membrane is minimized, and a structure in a state of having the membrane bonded thereto is firmly maintained so that the structure perform proper functions in a process of cell culture or cell research.

Effects of the present disclosure are not limited to the effects description described above, and other effects may be understood based on the following description, and clearly based on embodiments of the present disclosure.

### BRIEF DESCRIPTION OF THE DRAWINGS

FIG. 1 is a diagram illustrating a membrane bonded to various bonding bodies.

FIG. 2 is a diagram illustrating an example of using the membrane and a cell culture vessel which is the bonding body.

FIG. 3 illustrates a process of thermally bonding the membrane to the bonding body.

FIG. 4(a) a diagram illustrating a nanofiber structure of each of a bonding surface and a permeable surface of the membrane, and a boundary surface between the bonding surface and the permeable surface. FIG. 4(b) is a graph experimentally checking an aspect in which an optimal heating temperature needed for thermal bonding varies depending on a thickness of a nanofiber. FIG. 4C is a diagram illustrating forming of a preserved surface, a deformed surface, and the bonding surface.

FIG. 5 is a diagram for showing a comparison between a method of attaching the membrane to the bonding body in the related art and a method of thermally bonding the membrane and the bonding body according to the present disclosure through a drawing or a captured image.

FIG. 6 is a diagram showing [Experimental Example 1] of the present disclosure.

FIG. 7 is a diagram showing [Experimental Example 2] of the present disclosure.

FIG. 8 is a diagram showing [Experimental Example 3] of the present disclosure.

FIG. 9 is a detailed diagram illustrating a method of manufacturing the structure to which the membrane is thermally bonded according to the present disclosure.

FIG. 10 illustrates configuring of a bonding surface and a permeate surface to have a shape that a user arbitrarily desires.

FIG. 11 illustrates the structure to which the membrane is thermally bonded is impregnated in a hydrogel composition.

FIG. 12 illustrates a blood brain barrier (BBB) model using brain microvascular endothelial cells (BMECs) derived from induced pluripotent stem cells (iPSCs).

FIG. 13 is a diagram illustrating a structure mass production device according to another embodiment of the present disclosure.

### BEST MODE FOR CARRYING OUT THE INVENTION

Hereinafter, embodiments of the present disclosure are described in detail with reference to the accompanying drawings. Advantages and features of the present disclosure and methods of accomplishing the same may be understood more readily with by reference to the following detailed description of embodiments and the accompanying drawings. However, the present disclosure is not limited to the embodiments set forth herein, and may be embodied in many different forms. The embodiments are provided so that this disclosure will be thorough and complete, and will fully convey the concept of the present disclosure to those skilled in the art, and the scope of the present disclosure should be defined by the appended claims. Like reference numerals denote like elements throughout this specification.

Unless defined otherwise, all the terms (including technical and scientific terms) used in this specification may be used as meanings that may be commonly understood by those skilled in the art. In addition, terms defined in commonly used dictionaries are not interpreted ideally or excessively unless clearly and specially defined. As used herein, the term is intended to describe the embodiments but is not intended to limit the present disclosure. In this specification, the singular includes the plural unless specifically stated otherwise.

“While such terms as “first,” “second,” etc., may be used to distinguish one component from another component without departing from the scope of protection. For example, a first component may be referred to as a second component, and similarly, a second component may be referred to as a first component.

It will be further understood that the terms “comprises,” “comprising,” “includes,” and/or “including,” when used herein, mean that the mentioned components, steps, operations, and/or elements do not exclude the presence or addition of one or more other components, steps, operations and/or elements.

The present disclosure relates to a structure to which a nanomembrane is thermally bonded and a method of manufacturing the structure. The present disclosure is to resolve several problems (e.g., bonding separation, a leak, etc.) which occurred in general several methods of using adhesives or adhesive tapes, by thermally bond a porous nanomembrane 10 to a bonding body 20 to maximize adhesiveness (or bonding force) between the membrane 10 and the bonding body 20.

As briefly mentioned above in the background, a porous nanomembrane refers to a membrane formed when a plurality of polymer fibers are entangled with each other, and is a membrane having a thickness of several tens of nm to several tens of μm and including a plurality of permeation pores that selectively allow a particular substance to pass through.

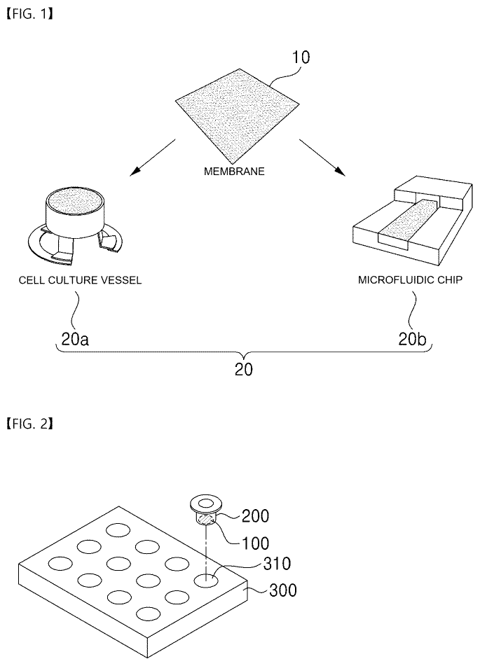

FIG. 1 is a diagram illustrating the membrane 10 bonded to various bonding bodies 20. Referring to FIG. 1, the membrane 10 may be bonded to a cell culture vessel 20a or a microfluidic chip 20b which is the bonding body 20.

In this detailed description, the bonding body 20 is mainly described, particularly, with reference to a cell culture vessel 20a among various bonding bodies 20. However, the bonding body 20 is not limited thereto. Any structure having a surface to which the membrane 10 may be bonded for cell culture, cell research, and analysis may be included within a scope of the bonding body 20. Alternatively, any type of structure having a surface to which the membrane 10 may be bonded may be included as a type of the bonding body 20 even when the structure is not related to cell culture, research, or analysis. A thermoplastic may be used as a material of the bonding body 20. Preferably, a plastic polymer used for injection processing such as polystyrene (PS), polymethylmethacrylate (PMMA), or cyclic olefin copolymer (CoC) may be used.

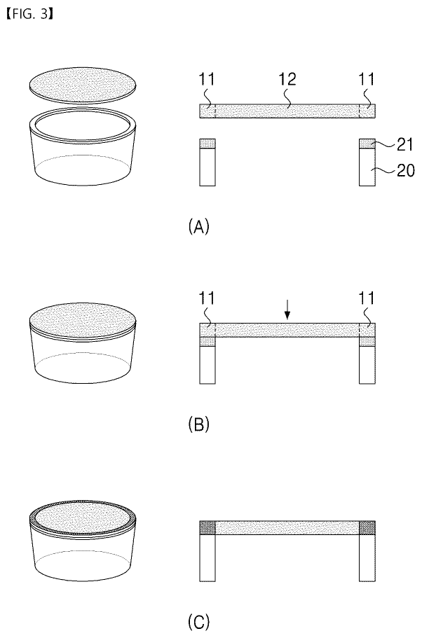

FIG. 2 is a diagram illustrating an example of using a membrane and a cell culture vessel which is a bonding body. FIG. 2 is a diagram for helping understanding of utilization of a structure for a membrane and a bonding body to be described in the

### DETAILED DESCRIPTION

Referring to FIG. 2, a membrane 100 is attached and bonded to one surface of a cell culture vessel 200, and the cell culture vessel 200 to which the membrane 100 is bonded may be inserted into an opening portion 310 of a plate 300.

As such, when a membrane is bonded to a cell culture vessel, permeation pores of the membrane allow oxygen and nutrients contained in a fluid to be supplied more smoothly to a cell side in the cell culture vessel, thereby inducing further promotion of cell proliferation and differentiation. At this time, the membrane may also function as a cell culture surface on which cells are cultured.

In addition, since the membrane includes a plurality of polymer nanofibers, the membrane may have a structure similar to that of an in vivo basement membrane, and thus may be utilized to implement an environment similar to an in vivo blood flow environment.

As such, the structure for by a membrane and a bonding body may be utilized to implement various environments for cell culture and analysis. A purpose of the present disclosure is to enable to easily create such a highly useful structure without defects.

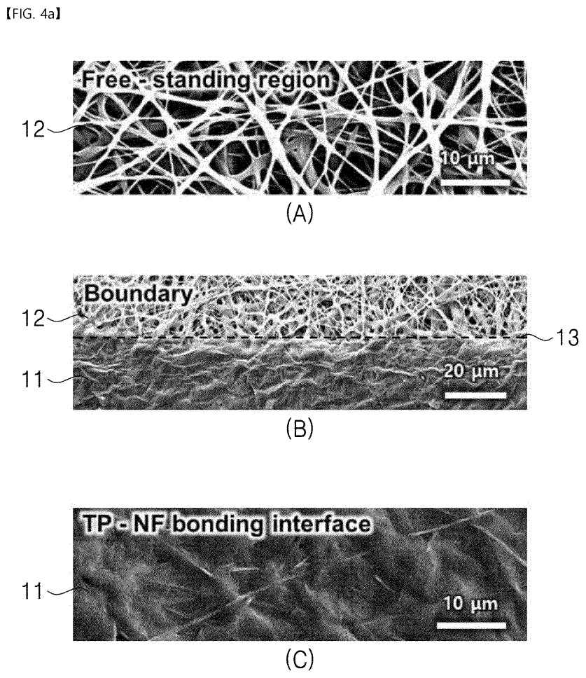

FIG. 3 illustrates operations of thermally bonding the membrane 10 to the bonding body 20.

Operation (A) in FIG. 3 includes partially applying heat to a bonding portion 21 of the bonding body 20 to thermally bond the membrane 10 to the bonding body 20 . . . . Here, the bonding portion 21 refers to a portion of a structure of the bonding body 20 directly in contact with the membrane 10. In FIG. 3, a mouth portion of the bonding body 20 having a cylindrical shape may correspond to the bonding portion 21. The bonding body 20 may be basically made of a heat-resistant material. In some cases, even when a whole structure of the bonding body 20 is not heat-resistant, at least the bonding portion 21 may be made of a heat-resistant material. In addition, the bonding portion 21 may have a shape closed by a closed curve in a planar view. As a result, a bonding surface 11 obtained through thermal bonding to the membrane 10 needs to have a closed shape so that a fluid permeation phenomenon is meaningful. Accordingly, the bonding portion 21 may have a closed shape in any way.

Meanwhile, the bonding surface 11 of the membrane 10 refers to a surface in direct contact with the bonding portion 21 of the bonding body 20, and a boundary may be present between the bonding surface 11 and the permeation surface 12, i.e., the permeation surface 12 configured to function for fluid permeation among surfaces of the membrane 10. Also, for reference, the bonding surface 11 has characteristics of significantly limiting passage of cells or fluids after thermal bonding, and may be understood as a concept including a region or a surface in which a gap is not present as a nanofiber structure is crushed and bonded to the bonding body. The significantly limiting of passage of cells (micro scale cells) or fluids means that passage of cells or fluids at the bonding surface 11 is limited to less than 5% compared to the permeation surface 12. However, it may be understood that a most desirable state of the bonding surface 11 is a state in which passage of cells or fluids is completely blocked, i.e., a permeability of 0%. Meanwhile, it may be also understood that when a temperature of the bonding portion 21 of the bonding body 20 is lower than a melting point of a membrane, bonding itself is not performed, and thus, the bonding surface 11 may not be created, and the bonding surface 11 may be created only in a state when the bonding portion 21 is heated to a temperature higher than the melting point of the membrane and bonded to the membrane.

As a reference, in the detailed description, a structure in which the membrane 10 is configured to be thermally bonded to the bonding body 20, i.e., a structure in which the bonding surface 11 of the membrane 10 is thermally bonded to the bonding portion 21 of the bonding body 20 is referred to as a ‘structure 1 to which the membrane 10 is thermally bonded.’

Referring back to FIG. 3, operation (B) after operation (A) in FIG. 3 includes causing the membrane 10 to approach and come into contact with the bonding portion 21 of the bonding body 20, to which heat is applied, and bonding the membrane 10 to the bonding portion 21. This operation may be implemented using a method of causing the bonding portion 21 of the bonding body 20 to approach the membrane 10 when the membrane 10 is fixed not to be moved, or a method of causing the membrane 10 to approach the bonding portion 21 when the bonding body 20 is fixed not to be moved, or a method of causing the membrane 10 and the bonding portion 21 to approach each other.

Meanwhile, in operation (B), the membrane 10 may approach and be bonded to the bonding portion 21 while maintaining a flat state, but is not limited thereto. In other words, the membrane 10 may approach and be bonded to the bonding portion 21 while maintaining a curved surface with a predetermined curvature value, instead of being in a flat state. The predetermined curvature value may be determined according to an intention of a designer of the structure. For example, (i) a relatively large curvature value may be determined when a nanomembrane filter in a sagging state is to be implemented to cultivate culture cells within a curved surface, or (ii) a relatively small curvature value may be determined to prevent durability of the permeation surface 12 from being reduced by fine shrinkage of a fiber structure when the fiber structure is hardened due to contact and bonding with the bonding portion 21. That is, even when a shrinkage phenomenon of a fiber structure occurs due to bonding, since the membrane 10 with a curvature value is used based on anticipation of the shrinkage phenomenon in advance, the permeation surface 12 having a flat surface may be resultantly obtained after bonding.

As a reference, the above-described embodiments (i) and (ii) refer to additional embodiments. It should be noted that a bonding process according to the present disclosure basically assumes an embodiment such that a membrane having a planar surface without a curvature is bonded to a bonding body.

On the other hand, in operation (B), a process of controlling a speed of approach between the membrane 10 and the bonding portion 21 may also be performed. Bonding is caused by a change in a fiber structure which occurs at time of contact between the bonding portion 21 and the membrane 10, or in some cases, before the contact occurs. At this time, a speed at which the membrane 10 and the bonding portion 21 approach each other may be adjusted from 0.1 mm/s to 100 mm/s so that a designer may manufacture a desired structure. A magnitude of a bonding speed may vary depending on an area of the bonding portion 21, a thickness of the membrane 10, or physical properties of the bonding portion 21.

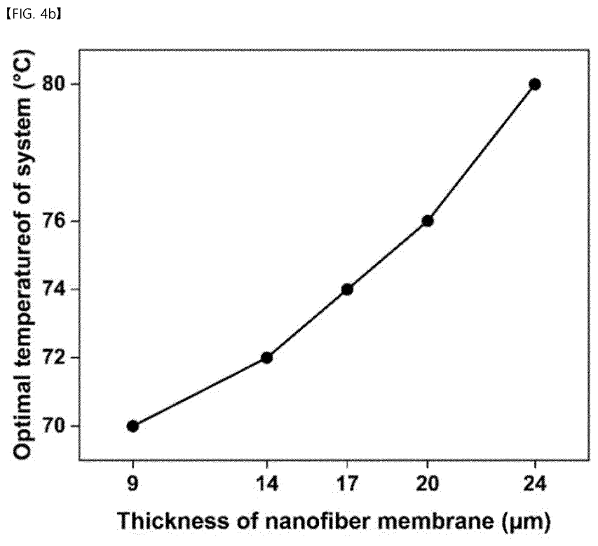

In some cases, even when a permeation surface needs to be adjusted at the permeation surface 12, an approaching speed may be controlled to implement an area of the permeation surface 12 according to a need by a designer (an experimenter). As will be explained in detail with reference to FIG. 4(c), the permeation surface 12 may be divided into a preserved surface 12a and a deformed surface 12b. The preserved surface 12a means a region of the permeation surface 12 which is not deformed by heat. On the other hand, the deformed surface 12b means a region deformed by heat. At this time, it is to be understood that the deformed surface 12b indicates a surface different from the bonding surface 11. In addition, it needs to be assumed that the bonding surface 11 is a surface in contact with the bonding portion 21. Meanwhile, when the permeation surface 12 may be divided into the preserved surface 12a and the deformed surface 12b, an area of a permeable surface (permeate surface) on the permeation surface 12 may be adjusted according to an approach speed. For example, when the deformed surface 12b of the permeation surface 12 is to be minimized, bonding may be performed by reducing an approach speed of at least one of the membrane 10 and the bonding portion 21 to be relatively small, that is, increasing approach time. At this time, a temperature of the bonding portion 21 may be relatively lower compared to a case when the deformed surface 12b is enlarged, which will be described later. On the other hand, when the deformed surface 12b of the permeation surface 12 is to be enlarged, the approach speed may be increased so that the bonding portion 21 in a heated state may come into contact with a surface of the membrane at a high temperature as possible to obtain a large area of the deformed surface. At this time, a temperature of the bonding portion 21 may be relatively higher compared to the cases described above so that sufficient bonding is obtained within a short period of time.

Lastly, after operation (B) in FIG. 3, operation (C) includes providing bonding between the bonding portion 21 and the bonding surface 11 according to thermal deformation and hardening of a nanofiber structure of the bonding surface 11 in contact with the bonding portion 21. In the drawing illustrating operation (C) in FIG. 3, the bonding portion 21 is not distinguished from the bonding surface 11. This is to emphasize that a structural transformation has occurred to a state in which the bonding portion 21 and the bonding surface 11 are completely bonded to each other not to be distinguished from each other, particularly, a state in which structural transformation of nanofibers constituting the membrane has occurred.

FIG. 4(a) is a diagram illustrating a nanofiber structure of each of the bonding surface 11 and the permeation surface 12 of the membrane 10, or a boundary surface 13 between the bonding surface 11 and the permeation surface 12.

(A) in FIG. 4(a) illustrates a nanofiber structure in a state when the membrane 10 is fixed for bonding, or the permeation surface 12 after bonding is performed but deformation of the fiber structure is not present. Referring to (A) in FIG. 4(a), it may be checked that the permeation surface 12 is configured to have a structure in which a plurality of polymer fibers are entangled with each other to include a plurality of permeation holes configured to selectively allow a particular substance to pass through. The permeation surface 12 having such characteristics is a surface actually utilized in an experiment, and thus, may generally occupy a relatively large area compared to the bonding surface 11. At this time, a diameter of nanofibers may be in a range from several tens of nanometers to several micrometers, and a number of micropores per unit volume may vary depending on a thickness. A thickness of the membrane 10 made of such nanofibers may be in a range from several hundred nanometers to several tens of micrometers. As a reference, a diameter of nanofibers may preferably be in a range from several tens of nm to 2000 nm, and a thickness of a nanofiber material may preferably be 3 μm or more. On the other hand, an optimal heating temperature of a bonding body for bonding may increase depending on a thickness of the nanofibers. For example, referring to FIG. 4(c), it may be understood that an optimal heating temperature (an optimal temperature of a system) tends to increase when a thickness of the nanofibers (a nanofiber membrane) increases.

Meanwhile, referring to (B) in FIG. 4(a), it may be checked that the boundary surface 13 may be defined between the permeation surface 12 and the bonding surface 11 after thermal bonding of the membrane 10 and the bonding body 20. Here, the boundary surface 13 is literally a surface for defining a boundary between the permeation surface 12 and the bonding surface 11. In some cases, the boundary surface 13 may function to block fluids (or fine substances) passing through the permeation surface 12 from being leaked or flowing out.

As a reference, the nanofiber structure of the boundary surface 13 may be partially crushed due to thermal deformation. However, since a remaining portion has permeability, the nanofiber structure may perform a barrier function as described above, and partially perform a function of the permeation surface 12 and the bonding surface 11.

Referring to (C) in FIG. 4(a), after thermal bonding of the membrane 10 and the bonding body 20, the nanofiber structure of the bonding surface 11 is crushed by thermal deformation and hardened again to be resultantly transformed into a state without permeation pores. The bonding surface 11 having such a transformed structure prevents cells or fluids accommodated in the bonding body 20 from leaking out of the boundary surface 13. The bonding surface 11 may only include a region in which a nanofiber structure clearly distinguishable compared to the permeation surface 12 is not present. Alternatively, even when a nanofiber structure is present, nanofibers having diameters of 200% or more larger than diameters of nanofibers in the permeate surface may be present. In addition, according to characteristics of the bonding surface 11 as described above, the boundary surface 13 between the permeation surface 12 and the bonding surface 11 may exhibit characteristics corresponding to a process in which the diameters of the nanofiber increase to 200% or more.

In this way, a function of preventing moving bodies such as cells accommodated in the bonding body 20 from moving out of the boundary surface 13 has a significant impact on measurement of results of cell culture and cell research/analysis. In detail, a main purpose of cell culture is to proliferate cells, but when cells escapes out of the bonding body 20, the main purpose may not be properly achieved, and poor experimental results may be obtained. However, technology of bonding the membrane 10 to the bonding body in the related art has utilized a method of applying an adhesive or attaching (taping) an attachment to the bonding body 20. This technology has not solve a problem such that cells escape from the bonding body 20. Accordingly, it should be emphasized that the present disclosure is to solve the problem described above by thermally bonding the membrane 10 to the bonding body 20.

FIG. 4(c) is a plan view of the membrane 10 on which the bonding has been performed. In the drawing, the bonding surface 11, the permeation surface 12, and the boundary surface 13 therebetween are shown. Further, it may be checked that the preserved surface 12a and the deformed surface 12b within the permeation surface 12 are illustrated. The permeation surface 12 does not necessarily include the deformed surface 12b, but may only include the preserved surface 12a without a deformation (damage) to the membrane, depending on a bonding method.

As a reference, the preserved surface 12a and the deformed surface 12b may be defined by a criterion described below.

For example, the deformed surface 12b may be defined as a surface having a light transmittance of 70% or more, and the preserved surface 12a may be defined as a remaining region. In a case of the deformed surface 12b, light transmittance or fluid permeability may be increased and a size of micropores may be increased, and rigidity of a nanofiber membrane increases. Thus, this may have a negative impact on cell culture. On the other hand, the deformed surface 12b may be defined as a region with a light transmittance of 90% or more, and the preserved surface 12a may be defined as a remaining region. In this way, the deformed surface 12b may be defined as a region with a relatively higher transmittance compared to the preserved surface 12a, as being divided with reference to a point where a transmittance is critically changed.

As another example, the preserved surface 12a and the deformed surface 12b of the membrane may be distinguished from each other by roughness. Generally, a region where nanofibers are deformed exhibits such characteristics that a structure in which nanofiber strands are stacked according to layers disappears and a surface becomes smooth. Considering such characteristics, the deformed surface 12b may be defined as a region in which, when viewed from a side cross-section in a state in which nanofibers are crushed, a thickness difference between a peak (a highest point of a nanofiber structure) and a valley (a lowest point of the nanofiber structure) is equal to or less than several tens of nm. Additionally, the preserved (non-deformed) surface 12a may be defined as a region other than the deformed surface 12b. As a reference, in a case of the preserved surface 12a, nanofiber strands may be maintained without any state change, and at this time, when viewed from a side cross-section, a thickness difference between a peak and a valley of nanofibers may be in a range from several hundreds of nm to several μm.

As another example, the preserved surface 12a and the deformed surface 12b of the membrane may be defined with reference to a ratio of diameters of nanofibers. In detail, the deformed surface 12b may be defined as a region in which a nanofiber diameter has increased by several tens to several hundreds of percent or more compared to a normal nanofiber diameter. The preserved surface 12a may be defined as a region other than the deformed surface 12b. As a reference, a range of a normal nanofiber diameter may mean a range within which diameters of nanofibers observed within a given arbitrary membrane may be included.

As another example, the preserved surface 12a may be defined as a surface in which nanofibers having a diameter in a range from several tens of nanometers to several micrometers are observed, and the deformed surface 12b may be defined as a remaining region.

As another example, the deformed surface 12b may be defined as a region in which a thickness of the membrane is reduced by 30% or more compared to the preserved surface 12a.

The membrane 10 after bonding has a planar structure as shown in FIG. 4(c). Generally, a case when proper bonding has been performed between the membrane 10 and the bonding body 20 is defined as a case when the deformed surface 12b takes up 1% or less of the whole permeation surface 12.

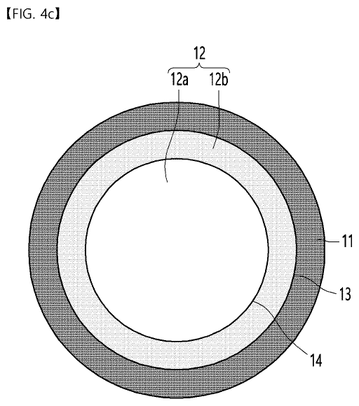

FIG. 5 is a diagram for showing a comparison between a method of attaching the membrane 10 to the bonding body 20 in the related art and a method of thermally bonding the membrane 10 and the bonding body 20 according to the present disclosure through a drawing or a captured image.

(A) in FIG. 5 and (B) in FIG. 5 show a comparison between a cell leakage in an experiment conducted using an adhesive tape in the related art ((A) in FIG. 5) and a cell leakage when a thermal bonding method according to the present disclosure is used ((B) in FIG. 5).

Comparing (A) in FIG. 5 and (B) in FIG. 5, it may be understood that in a method of using an adhesive tape in the related art, some cells have breached a boundary surface and leaked out, and in the thermal bonding method according to the present disclosure, cells are present only inside the permeation surface 12 within the boundary surface 13. As a reference, in the method of using an adhesive tape in the relation art, the cells having breached the boundary surface and leaked out cannot be properly supplied with nutrients or oxygen. Thus, desired experimental data regarding the corresponding cells may not be obtained, and overall cell culture may not be performed properly.

(C) in FIG. 5 and (D) in FIG. 5 are drawings exemplarily illustrating cell leakage in the method of using an adhesive tape in the related art ((C) in FIG. 5) and cell leakage in the thermal bonding method according to the present disclosure ((D) in FIG. 5A).

As shown in (C) of FIG. 5, in the method of using an adhesive tape in the related art, even when the bonding body 20 and the membrane 10 are attached to each other via an adhesive tape, the cells may leak out via a gap in an adhesive surface 11a.

On the other hand, referring to (D) in FIG. 5, in the thermal bonding method according to the present disclosure, the cells are prevented from leaking due to bonding between nanofibers of the membrane (10) and the bonding body 20 due to thermal deformation. Thus, the cells may only move within the permeation surface 12, thereby obtaining high-quality experimental data and an excellent cell culture effect.

As such, it may be understood that a nanofiber structure of the membrane 10 thermally deformed by thermal bonding may prevent cell leakage, and further, function as an important factor for obtaining good results in cell culture and cell research/analysis.

Meanwhile, to constitute the nanofiber structure of the membrane 10, the bonding portion 21 needs to be heated to a ‘particular temperature.’ A range from [a melting point of the membrane] to [the melting point of the membrane+10 °C.] may be defined as a preferred range of the particular temperature range. A melting point of a nanofiber material needs to be lower than a melting point of a bonding body or glass transition temperature. Considering that a glass transition temperature of polystyrene is approximately 100 degrees Celsius and that of polymethylmethacrylate (PMMA) is approximately 116 degrees Celsius, it is desirable to use a nanofiber material with a melting point of 120 degrees Celsius or lower.

For example, when the membrane 10 including nanofibers and made of polycaprolactone (PLC) is thermally bonded, a ‘range of a particular temperature’ may be from 62 ° C to 68 ° C, and a value of the particular temperature may be preferably determined as 65 °C.

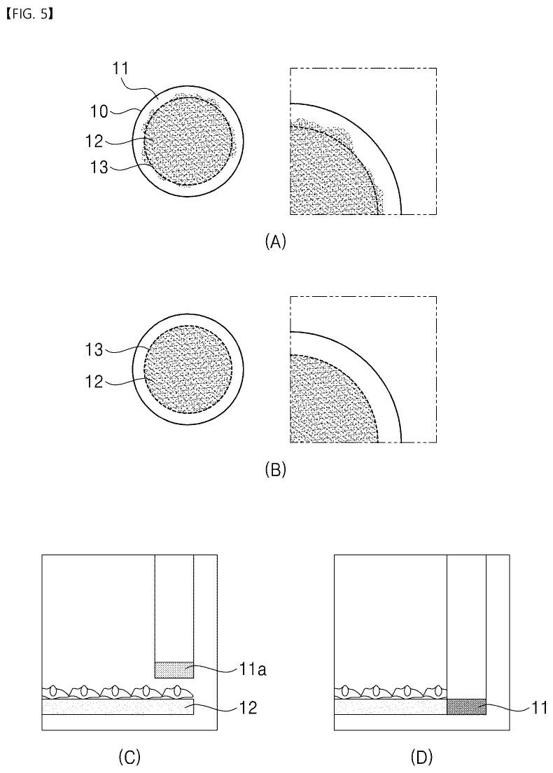

A basis for which a nanofiber structure of the membrane 10 optimized for thermal bonding may be constituted at the above-described temperature is to be described through [Experimental Example 1], [Experimental Example 2], and [Experimental Example 3] of FIGS. 6 to 8.

As a reference, it has been described above that a temperature of the bonding portion 21 at a moment when the bonding portion 21 comes into contact with the membrane is an important factor in determining a state of a bonding surface, and when this temperature is in a range from [a melting point of the membrane] to [the melting point of the membrane+10 °C.] (for convenience, this temperature range is referred to as a contact temperature range), a bonding surface, a permeate surface, and a boundary surface each desired by a user (an experimenter) may be defined. However, at this time, the bonding portion 21 may be heated to a temperature higher than the contact temperature range (for convenience, referred to as a preset temperature) before coming into contact with the membrane. This takes into account a situation in which the bonding portion 21 is naturally cooled during time for which the bonding portion 21 come into contact with the membrane. The preset temperature may be determined by taking into account time or a cooling speed from a time point when the bonding portion 21 is heated to a time point when the bonding portion 21 comes into contact with the membrane. The preset temperature may preferably 20 ° C higher than the contact temperature range.

FIG. 6 is a diagram showing [Experimental Example 1] of the present disclosure.

### Experimental Example 1

(A) in FIG. 6 shows a graph showing a change in temperatures of the bonding surface 11 and the permeation surface 12 of the membrane 10 depending on a temperature applied to the bonding portion 21 of the bonding body 20. This graph shows Tm-PCL of 60 ° C (hereinafter referred to as a melting point) as a reference line indicating a temperature at which a nanofiber structure is deformed. In [Experimental Example 1] of the present disclosure, the melting point is utilized to analyze data. The melting point is used to derive a temperature at which a nanofiber structure of the membrane 10 optimized for thermal bonding is provided.

As a reference, it may be understood that a temperature of the bonding surface 11 is different from that of the permeation surface 12 during thermal bonding because the bonding surface 11 is in direct contact with the bonding portion 21 of the bonding body 20 which is heated by heat, and thus, has a higher temperature compared to the permeation surface 12, whereas the permeation surface 12 has a lower temperature compared to the bonding surface 11 as being adjacent to, but not in direct contact with the bonding portion 21.

Referring to (A) in FIG. 6, temperatures Tₚₛ applied to the bonding body 20, more precisely, the bonding portion 21 are 55 ° C, 60 ° C, 65 ° C, 70 ° C, and 75 ° C, and temperatures of the bonding surface 11 and the permeation surface 12 are monitored. It is shown that when the bonding portion 21 is heated to 55 ° C and 60 ° C, temperatures of both the bonding surface 11 and the permeation surface 12 do not reach T_{m-PCL}. Conversely, when the bonding portion 21 is heated to 70 ° C and 75 ° C, it is found that temperatures of both the bonding surface 11 and the permeation surface 12 exceed T_{m-PCL}, showing significant deformation of a nanofiber structure. Meanwhile, when the bonding portion 21 is heated to 65 ° C, a temperature of the bonding surface 11 exceeds T_{m-PCL}, Whereas a temperature of the permeation surface 12 does not exceed T_{m-PCL}. Therefore, it is found that the nanofiber structure is maintained in a most ideal state.

Meanwhile, (B) in FIG. 6 shows actual photographs of nanofiber structures of the bonding surface 11 and the permeation surface 12, as affected by a heating temperature.

Referring to (B) in FIG. 6, when a ‘temperature Tₚₛ applied to the bonding body 20’ is 55 ° C, the bonding surface 11 does not properly melt, and thus, only a portion of a nanofiber structure is deformed. In addition, it is shown that at this temperature, thermal bonding between the membrane 10 and the bonding body 20 is not properly performed, and even when the bonding is partially performed, a bonding strength between the membrane 10 and the bonding body 20 is weak, resulting in easy separation therebetween and occurrence of cell leakage.

When the ‘temperature Tₚₛ applied to the bonding body 20’ is 65 ° C, a nanofiber structure of the bonding surface 11 is deformed by heat to have a bonding strength with the bonding body 20. At this time, a nanofiber structure of the permeation surface 12 is not thermally deformed, and thus, exhibits an optimal fiber structure for thermal bonding.

Meanwhile, when the ‘temperature Tₚₛ applied to the bonding body 20’ is 75 ° C, it is found that the nanofiber structure of the bonding surface 11 is deformed by heat, and a part of the permeation surface 12 adjacent to the bonding surface 12 is also thermally deformed, and thus, a permeation function of the permeation surface 12 may be partially lost. Through the [Experimental Example 1] described above, it is found that the ‘particular temperature’ for forming the nanofiber structure of the membrane 10 in thermal bonding is 65 ° C, and that when thermal bonding was performed at less than 75 ° C, the normal structure 1 subject to thermal bonding may be obtained as a result. Thus, it may be checked that a temperature needed for thermal bonding is within a range from a melting point of the membrane 10 to a value 10 ° C higher than the melting point of the membrane 10.

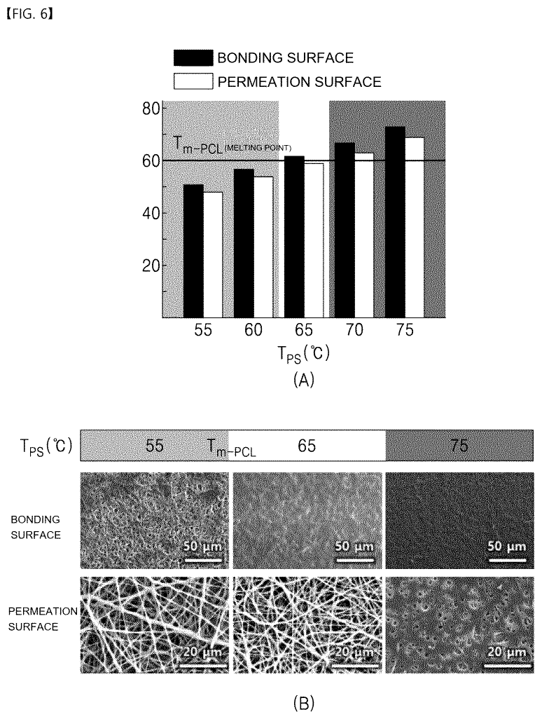

FIG. 7 is a diagram showing [Experimental Example 2] of the present disclosure.

### Experimental Example 2

[Experimental Example 2] of the present disclosure is a drawing showing, in color, a heat conduction phenomenon during a process of thermal bonding between the membrane 10 and the bonding body 20.

(A) to (C) in FIG. 7 show a heat conduction phenomenon between the membrane 10 and the bonding body 20 when ‘temperatures Tₚₛ applied to the bonding body 20’ are 55 ° C, 65 ° C, and 75 ° C, respectively. In addition, in (A) to (C) in FIG. 7, a temperature at a corresponding location is indicated by color, and a temperature of the corresponding color is indicated on a right side (e.g., dark red=80 ° C, dark blue=24 ° C)

(A) in FIG. 7 shows a case when a ‘temperature Tₚₛ applied to the bonding body 20’ is 55 °C. In this case, it may be inferred that the bonding surface 11 has not yet reached a temperature (60 ° C.: yellow) corresponding to a range of a melting point, and thus thermal deformation has not occurred properly.

(B) in FIG. 7 shows a case when a ‘temperature Tₚₛ applied to the bonding body 20’ is 65 °C. In this case, it may be understood that the bonding surface 11 reaches a range of a melting point, and thus, thermal deformation occurs and, simultaneously, a phenomenon of bonding between the membrane 10 and the bonding body is caused. Meanwhile, it may be understood that since a temperature conducted to the permeation surface 12 by heat is in a range equal to or below the melting point (yellow), thermal deformation may not occur in the permeation surface 12

(A) in FIG. 7 shows a case when a ‘temperature (Tₚₛ) applied to the bonding body 20’ is 75 °C. In this case, the bonding surface 11 will reach a range exceeding the melting point, and thus, thermal deformation may occur, and it may be understood that a temperature equal to or higher than the melting point is transferred to the permeation surface 12 adjacent thereto due to a heat conduction phenomenon, and thermal deformation may be caused.

Through the [Experimental Example 2] described above, it is found that at ‘a particular temperature’ of 65 ° C in the present disclosure, thermal deformation occurs only on the bonding surface 10 and a thermal conduction phenomenon occurs according to a transfer of a temperature below the melting point (60 ° C.: yellow), and thus, an optimal nanofiber structure of the membrane 10 without thermal deformation of the permeation surface 12 may be provided.

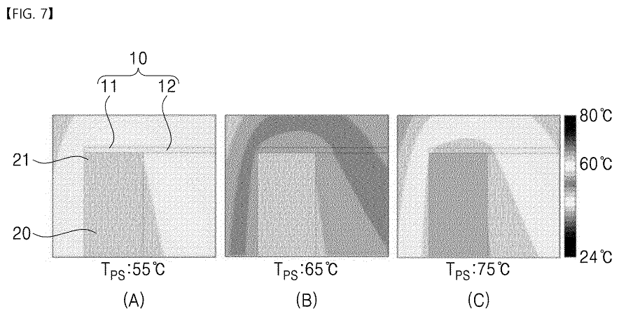

FIG. 8 is a diagram showing [Experimental Example 3] of the present disclosure.

### Experimental Example 3

[Experimental Example 3] of the present disclosure further supports that the ‘particular temperature’ of 65 ° C, i.e., a same temperature as the melting point of the membrane is an optimum temperature. After heating the bonding portion 21 to a temperature of 55 ° C, 65 ° C, and 75 ° C, respectively, actual results when the membrane 10 is thermally bonded are compared to each other. Then, based on the actual results, a range of temperatures at which the permeation surface 12 is not deformed when heated to the temperatures described above is shown in a graph.

First, (A) in FIG. 8 shows a case when heat of 55 ° C is applied to the bonding portion 21 of the bonding body 20 during thermal bonding. The structure at this time exhibits characteristics in which the bonding surface 11 shows a relatively bright color, in other words, deformation of a fiber structure has rarely occurred.

(B) in FIG. 8 shows a case when heat of 65 ° C is applied to the bonding portion 21 of the bonding body 20 during thermal bonding. Referring to (B) in FIG. 8, in the structure at this time, the bonding surface 11 has changed to a relatively darker color compared to (A), in other words, showed deformation of the fiber structure. In addition, a boundary between the bonding surface 11 and the permeation surface 12 was clearly shown. Particularly, in an region of the permeation surface 12, deformation of the fiber structure has not occurred, and a bright color is maintained.

(A) in FIG. 8 shows a case when heat of 75 ° C is applied to the bonding portion 21 of the bonding body 20 during thermal bonding. It may be checked that a portion of the permeation surface 12 which was to be placed on the bonding body 20 is subject to heat of a temperature above the melting point, and thus, thermally deformed. That is, it may be checked that a ratio of a region that may be utilized as the permeation surface 12, i.e., ‘a preserved permeation surface (a Y-axis in (D) in FIG. 8)’ has decreased.

Meanwhile, (D) in FIG. 8 is a graph showing a ‘ratio of a preserved permeation surface (a ratio of a normal permeation surface 12)’ of the permeation surface 12 depending on an applied temperature.

Referring to (D) in FIG. 8, it may be checked that when a temperature exceeding 65 ° C is applied, heat of a temperature above the melting point is conducted to the permeation surface 12, and thus, a surface that is not deformed by thermal deformation is gradually decreased and a ‘ratio of a preserved permeate surface’ is decreased, a surface preserved according to an increasing temperature is gradually decreased, and the preserved permeate surface is decreased to approximately 10% at 85 °C.

[Experimental Example 3] described above may support the experimental results of [Experimental Example 2]. In addition, it may be checked that when heat higher than a melting point of a membrane is applied to a bonding portion during thermal bonding, the heat is also transferred to the permeation surface 12, and a ratio of a preserved surface of the permeation surface 12, in other words, a preserved surface 12a is gradually decreased.

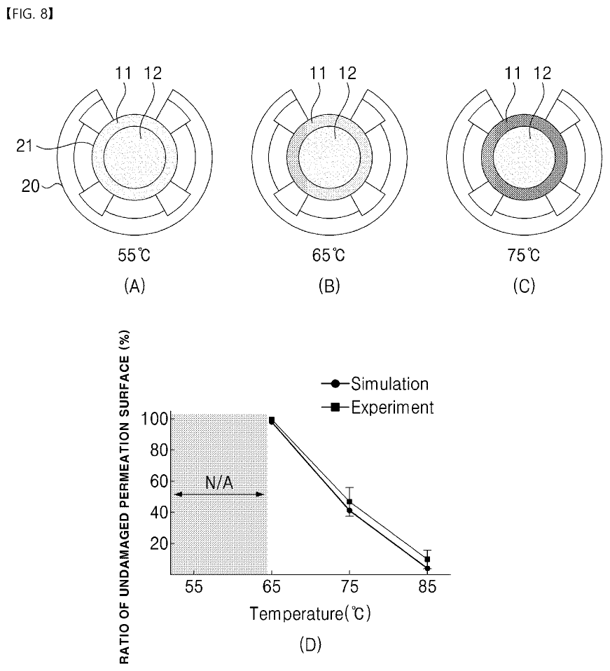

FIG. 9 is a diagram illustrating a method of manufacturing the structure 1 to which the membrane 10 in the present disclosure is thermally bonded.

Referring to FIG. 9, the method of manufacturing the structure 1 according to the present disclosure may include applying heat at a particular temperature to the bonding portion 21 of the bonding body 20 (S101). Here, the bonding portion 21 of the bonding body 20 refers to a surface bonded to correspond to the bonding surface 11 of the membrane 10 when the membrane 10 is bonded to the bonding body 20. In addition, the particular temperature described herein refers to a temperature at which, as the bonding surface 11 reaches a melting point at which the membrane 10 begins to be thermally deformed, heat equal to or less than the melting point is conducted to the permeation surface 12 adjacent to the bonding surface 11 in a thermal bonding process.

After operation S101, bonding both the bonding portion 21 and the membrane 10 by causing the bonding portion 21 of the bonding body 20, subject to the heat, to approach the membrane 10 (S102) may be included. At this time, bonding efficiency may be improved by applying a predetermined pressure (pressing) in a direction of the bonding.

Although not shown in FIG. 9, after operation S102, pausing a state of the bonding for a predetermined time may be added to allow the bonding surface 11 to be thermally deformed, and then, hardened.

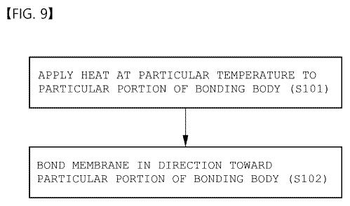

FIG. 10 illustrates configuring of the bonding surface 11 and the permeation surface 12 to have a shape that a user arbitrarily desires.

The user may configure the permeation surface 12 or the bonding surface 11 having a shape that the user arbitrarily desires. To configure the permeation surface 12 or bonding surface 11 to have a particular shape (e.g., a shape such as a triangle, a square, or a star) after thermal bonding, the user may configure a shape desired by the user on one surface of a heating body 30 (e.g., engraving), apply heat to the one surface of the heating body 30 including the configured shape, and then, press the one surface onto a surface of the membrane 10 like a stamp to obtain the permeation surface 12 or the bonding surface 11 having the desired shape.

Meanwhile, with respect to the structure 1 created through the process described above, subsequently bringing the structure 1 into contact with a hydrogel or a solution to coat nanofibers constituting the structure 1 with the hydrogel or the solution may be further performed so that the permeation surface 12 of the structure 1 performs a function similar to characteristics of extracellular matrix in an actual body. Preferably, the structure 1 may be impregnated into the hydrogel to bind the nanofibers with the hydrogel. In this detailed description, an embodiment in which the structure 1 is impregnated with hydrogel is described to help understanding of the disclosure. Porous membranes made of general plastic materials have significantly different forms and physical properties from those of the extracellular matrix, and thus, have limitations of considerable lack in similarity to in vivo cells in terms of forms and functions during cell culture. To overcome the limitations, a hydrogel having forms and physical properties similar to those of the extracellular matrix may be utilized. In a structure having been impregnated with a hydrogel (hereinafter referred to as a hydrogel structure), cells may grow based on physical/chemical structures and stimuli similar to those in a body. Resultantly, the hydrogel structure may function as a means to allow expression of a shape and a function of cells to be more similar to those in a body.

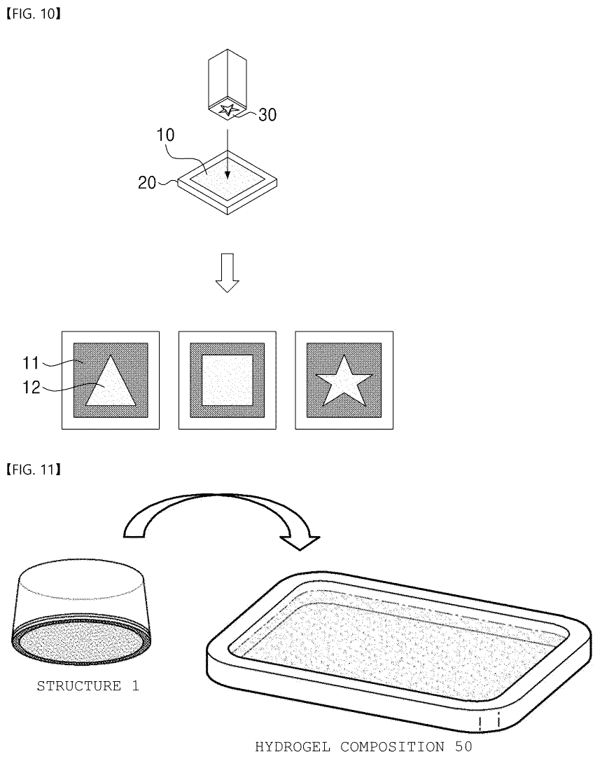

FIG. 11 illustrates the structure 1 to which the membrane is thermally bonded is impregnated in with a hydrogel composition 50. As shown in the drawing, when the hydrogel structure 50 is placed, the permeation surface 12 of the structure 1 may be sufficiently immersed in the hydrogel structure 50 to impregnate a membrane region of the permeation surface 12. At this time, the permeation surface 12 may have a mechanical strength of several tens of KPa to several GPa depending on characteristics of a nanofiber structure. Due to such characteristics, a hydrogel having with a weak mechanical strength may be impregnated with nanofibers, resulting in a hydrogel culture environment with an excellent mechanical strength.

Meanwhile, the nanofibers described in this detailed description may include at least one polymer nanofiber selected from the group consisting of silk fibroin, collagen, gelatin, cellulose, alginate, polycaprolactone, polyurethane, polyvinylidene fluoride (PVDF), polystyrene, polycarbonate, polyethylene terephtalate (PET), polytetrafluoroethylene (PTFE), polylactic acid, PLA polyvinyl alcohol (PVA), polymethacrylate (PMMA), polyethylene oxide (PEO), nylon-4, 6, polyglycolic acid (PGA), and poly(lactic-co-glycolic acid) (PLGA). Preferably, the nanofibers may include at least one polymer nanofiber selected from a bio-derived material such as silk fibroin, collagen, and gelatin preferably polymer nanofibers of silk fibroin. The nanofibers may include preferably at least one polymer nanofiber selected from bio-derived materials such as silk fibroin, collagen, or gelatin. More preferably, the nanofibers may include a polymer nanofiber made of silk fibroin. When silk fibroin is used, there may be an advantage of a strong binding strength with a hydrogel.

Meanwhile, the hydrogel may include at least one thin film component selected from the group consisting of collagen, gelatin, agarose, extracellular matrix (ECM)-derived materials, Matrigel, and Geltrex, and may be, preferably, collagen. When cells are cultured on a hydrogel containing collagen which constitutes a large portion of extracellular matrix components, there are advantages of improvements in cell survival rates, substrate adhesion, functionality, and the like. For example, a hydrogel composition may include 1 to 20 wt % of the thin film component and 80 to 99 wt % of phosphate buffered saline (PBS) or a cell culture medium. More preferably, the hydrogel composition may contain 3 to 30 wt % of the thin film component and 70 to 97 wt % of PBS or a cell culture medium. At this time, in addition to the thin film component, water, PBS, a cell culture medium, or an acid/base solution may be further included. In detail, the additional component described above may be included so that pH of the hydrogel composition becomes 5 to 9. When collagen is contained in a hydrogel thin film, a three-dimensional structure may be included when the pH is at a level of 7. Thus, a hydrogel thin film having a structure suitable for three-dimensional cell culture may be provided.

In addition, the hydrogel structure according to the present disclosure may have a three-dimensional structure since the permeation surface 12 is impregnated into the hydrogel. Accordingly, cells may be cultured in a three-dimensional structure of the permeation surface. As described above, since the hydrogel structure has excellent transparency compared to general cell culture membranes, cells may be easily observed during cell culture.

In addition, cells that may be cultured on the hydrogel structure according to the present disclosure may be fibroblasts, vascular endothelial cells, smooth muscle cells, nerve cells, cartilage cells, bone cells, skin cells, Schwann cells, or stem cells, but are not limited thereto.

Meanwhile, after the structure 1 is immersed in the hydrogel composition to impregnate the permeation surface 12, drying may be further performed. Time for the dying may be at least 10 minutes, for example, at least 30 minutes to up to 6 hours at a temperature of 20 to 50 °C. When the drying time is less than 30 minutes, a hydrogel may not be sufficiently dried, and a problem such that a membrane form of the permeation surface 12 is not properly formed may occur. However, the drying time is not limited thereto, and may be adjusted depending on a drying method. The drying may be performed using, for example, natural drying, oven drying, hot air drying, etc., but is not limited thereto. Preferably, the drying may be performed by natural drying.

On the other hand, before the coating of the structure 1 by bringing the structure 1 into contact with a hydrogel or a solution, modifying for changing properties of the nanofibers constituting the structure, e.g., hydrophilicity/hydrophobicity, cell adhesion/detachment characteristics, or biomolecule binding strength may be further performed.

Meanwhile, by using the hydrogel structure according to the present disclosure, a brain blood vessel cell barrier model having a shape and a function similar to those of a brain blood vessel tissue in a body may be created. As briefly described above, the hydrogel structure may create an environment similar to an actual cell production environment in a human body. This may be identically applied to culture of brain blood vessel cells. That is, the hydrogel structure according to the present disclosure may provide rigidity and ease of observation suitable for culturing brain blood vessel cells. An organelle structure, arrangement, and a shape of the brain blood vessel cells cultured on the hydrogel structure may be induced to be similar to those of brain blood vessel cells in a body, thereby allowing construction of an in vitro brain blood vessel cell barrier model having a physiologically similar level of barrier functions and metabolic capabilities. As a result, in fields where in vitro brain blood vessel models are used, such as brain disease drug evaluation and brain physiology research, an effect of simulation, observation, and analysis, in an in vitro circumstance, of phenomena similar to drug absorption and metabolism in actual in vivo brain blood vessels may be obtained.

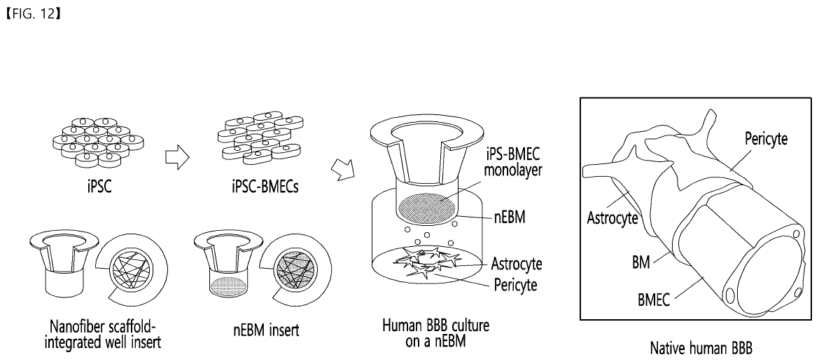

FIG. 12 illustrates a blood brain barrier (BBB) model using brain microvascular endothelial cells (BMECs) derived from induced pluripotent stem cells (iPSCs). Referring to the drawing, it may be checked that an entire culture system may be configured to have a hydrogel structure in an upper portion and a culture plate in a lower portion, and at this time, brain blood vessel cells differentiated from the induced pluripotent stem cells (iPS) may be cultured on the permeation surface 12 of the hydrogel structure bound with the upper portion, and astrocytes/pericytes may be cultured on a lower culture plate to constitute a brain blood vessel barrier model simulating a brain blood vessel structure in a human body.

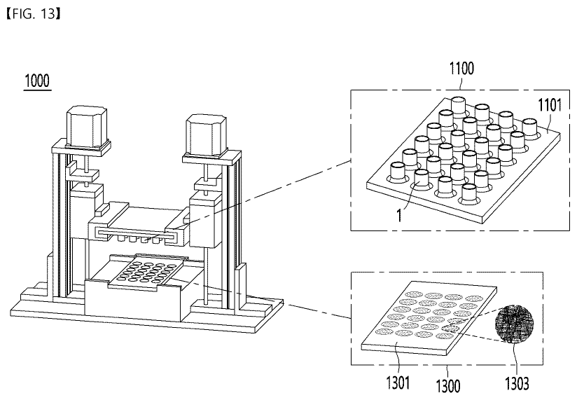

FIG. 13 is a diagram illustrating a structure mass production device 1000 according to another embodiment of the present disclosure. The structure 1 described in the detailed description may be mass-produced using a device as shown in the drawing. The structure mass production device 1000 may essentially include a structure array 1100 and a membrane array 1300.

The structure array 1100 may be configured to have a plurality of structures 1 provided on a first panel 1101. A bonding portion of each of the structures 1 may be heated by a heating element (not shown) separately provided in the structure mass production device 1000.

The membrane array 1300 is configured to have a plurality of membrane regions 1301 disposed on a second panel 1301. Each of the membrane regions 1301 may be configured to correspond to a size of the structures 1. There is no limitation on a manufacturing method or a detailed structure of the membrane array 1300, when a plurality of membrane regions 1301 are included in the membrane array 1300. For example, the second panel 1301 may be a thick case having a plurality of holes disposed therein. The second panel 1301 may be configured to have one complete nanofiber surface embedded therein so that a portion of the nanofiber surface is exposed through the plurality of holes. As another example, the second panel 1301 may be a hexahedron having a small thickness and constituted by having a plurality of nanofiber surfaces uniformly arranged on one side surface. It may be understood that the membrane array 1300 may be configured in various other methods.

The structure mass production device 1000 enables mass production of the structures 1 by heating the plurality of structures 1 on the structure array 1100, and then, immediately bringing the membrane regions 1301 on the membrane array 1300 and the structures 1 to approach each other to perform thermal bonding. The drawing shows a device designed such that the membrane array 1300 is arranged on a bottom surface and the structure array 1100 descends from above. However, there is no limitations on a structure of the device when heated bonding bodies of the structures 1 are designed to approach membrane regions.

Embodiments of the structure 1 to which the membrane 10 is thermally bonded and the method of manufacturing the structure, and a hydrogel structure in which the structure is impregnated in a hydrogel, and a method of constitute a brain blood vessel barrier model using the hydrogel structure have been described.

The present disclosure is not limited to the particular embodiments and application examples described above, and various changes and modifications thereof may be made by those skilled in the art without departing from the essence of the disclosure as claimed in the claims. Such changes and modifications should not be understood as being separate from the technical idea or viewpoint of the present disclosure.

| | |
|---|---|
| 1: Structure | 20b: Microfluidic Chip |
| 10: Membrane | 21: Bonding portion |
| 11: Bonding surface | 30: Heating body |
| 12: Permeation surface | 100: Membrane |
| 13: Boundary surface | 200: Cell culture vessel |
| 20: Bonding body | 300: Plate |
| 20a: Cell culture vessel | 310: Opening portion |

1. A structure to which a membrane is bonded, the structure comprising:
the membrane made of a material of a nanofiber; and
a bonding body which is a bonding target of the membrane,
wherein the membrane comprises:
a bonding surface constituted by being brought into contact with a bonding portion of the bonding body to which heat at a particular temperature is applied, and bonded to the bonding body by being thermally deformed due to the contact; and
a permeation surface other than the bonding surface, the permeation surface having a porous structure.

2. The structure of claim 1, wherein the particular temperature is in a range from a melting point Tm of the membrane to the melting point Tm of the membrane+10 °C.

3. The structure of claim 2, wherein the bonding surface is a surface of which a permeability is reduced due to deformation of a structure of the nanofiber compared to a permeability function before the thermal deformation, and through which cells of fluids, or fluids are significantly limited from passing.

4. The structure of claim 3, wherein the permeation surface comprises:
a surface deformed by heat; and
a surface preserved from heat.

5. The structure of claim 4, wherein the deformed surface is a region having a higher light transmittance compared to light transmittance of the preserved surface.

6. The structure of claim 4, wherein the deformed surface and the preserved surface are distinguished from each other based on roughness,
the deformed surface is a region in which a thickness difference between a peak and a valley both defined by the structure of the nanofiber is equal to or less than several tens of nm when a side cross-sectional surface of the membrane is observed, and
the preserved surface is a remaining area of the permeation surface, other than the deformed surface.

7. The structure of claim 4, wherein the deformed surface and the preserved surface are distinguished from each other based on a ratio of a diameter of the nanofiber,
the deformed surface is defined as a region in which a diameter having a value increased by a preset value or higher is observed, compared to any diameter of the diameter observed in the membrane in a state when heat is not applied, and
the preserved surface is a remaining area of the permeation surface, other than the deformed surface.

8. The structure of claim 4, wherein the deformed surface is a region in which a thickness of the membrane is reduced by a preset value or more compared to the preserved surface.

9. A method of manufacturing a structure to which a membrane is bonded, the method comprising:
(a) applying heat at a particular temperature to a bonding portion of a bonding body; and
(b) thermally bonding the bonding portion of the bonding body and the membrane by causing the bonding portion of the bonding body and the membrane to approach each other,
wherein the membrane after the thermally bonding comprises a bonding surface and a permeation surface,
the bonding surface is a surface constituted by being brought into contact with a bonding portion of the bonding body to which heat at a particular temperature is applied, and bonded to the bonding body by being thermally deformed due to the contact, and
the permeation surface is a surface other than the bonding surface, and has a porous structure.

10. The method of claim 9, wherein the permeation surface comprises a surface deformed by heat and a surface preserved by heat, the deformed surface and the preserved surface having values of different ratios between the deformed surface and the preserved surface depending on a speed at which the bonding portion and the membrane are caused to approach each other in operation (b).

11. The method of claim 9, wherein operation (a) comprises applying heat at a preset temperature having a value higher than a contact temperature range comprised in a range from a melting point Tm of the membrane to the melting point Tm of the membrane+10 °C.

12. The method of claim 9, further comprising, after operation (b), coating a nanofiber constituting the structure by bringing the structure to which the membrane is thermally bonded into contact with a hydrogel or a solution.

13. The method of claim 12, further comprising, before the coating of the nanofiber, modifying surface properties of the nanofiber constituting the structure.
